(19) United States
(12) Patent Application Publication
(10) Pub. No.: US 2002/0076378 A1
(43) Pub. Date: Jun. 20, 2002

(54) Polymers

(75) Inventors: Henry Wolfe, Wayne, PA (US); Kenneth Kellar, Wayne, PA (US)

Correspondence Address: Richard E. Fichter, BACON & THOMAS, PLLC, Fourth Floor, 625 Slaters Lane, Alexandria, VA, 22314-1176, US

(73) Assignee: NYCOMED IMAGING AS

(21) Appl. No.: 10/057,943

(22) Filed: Jan. 29, 2002

Related U.S. Application Data
(63) Continuation of application No. 09/362,711, filed on Jul. 29, 1999.
Application No. 09/362,711 is a continuation of application No. PCT/GB98/00270, filed on Jan. 29, 1998.
(60) Provisional application No. 60/057,704, filed on Aug. 27, 1997.

(30) Foreign Application Priority Data
Jan. 29, 1997 (GB) 9701813.9

(51) Int. Cl. A61K 49/04

(52) U.S. Cl. 424/9.4

(57) ABSTRACT

The invention provides compounds comprising a linear, branched or dendrimeric polymer backbone with linked thereto at least one reporter moiety, said polymer backbone comprising a plurality of amine-containing acids. Such compounds may be linked to one or more targeting agents and are useful as therapeutic and diagnostic agents, in particular in medical imaging techniques.

The present invention relates to polymers useful as therapeutic and diagnostic agents and to processes for their preparation. In particular, the invention relates to amino acid based biodegradable polymers for use in targeting of diagnostic imaging and therapeutic agents.

The polymers in accordance with the invention are suitable for use in a variety of applications where specific delivery is desirable, and are particularly suited for the delivery of biologically active agents. However, a preferred use of the polymers of the invention is in the enhancement of images of selected mammalian organs, tissues and cells in vivo using MR, X-ray, ultrasound, light and nuclear imaging techniques by virtue of their enhanced imaging properties and site specificity. The polymers are especially suited for use as intravascular contrast agents and blood pool agents in such imaging techniques. As such they may be used in imaging blood vessels, e.g. in magnetic resonance angiography, in the measurement of blood flow and volume, in the identification and characterization of lesions by virtue of differences in vascularity from normal tissue, in the imaging of the lungs for the evaluation of pulmonary disease and in blood perfusion studies.

Medical imaging techniques, such as MRI and X-ray, have become extremely important tools in the diagnosis and treatment of disease. Some imaging of internal parts relies on inherent attributes of those parts, such as bones, to be differentiated from surrounding tissue in a particular type of imaging, such as X-ray. Other organs and anatomical components are only visible when specifically highlighted by particular imaging techniques.

One such technique with the potential to provide images of a wide variety of anatomical components involves biotargeting image-enhancing metals. Such a procedure has the possibility of creating or enhancing images of specific organs and/or tumors or other such localized sites within the body, while reducing the background and potential interference created by simultaneous highlighting of non-desired sites.

It has been recognized for many years that chelating various metals increases the physiologically tolerable dosage of such metals and so permits their use in vivo to enhance images of body parts. One chelate complex which has been the subject of much study is Gd-DTPA. However, despite its satisfactory, relaxivity and safety, this has several disadvantages. Due to its low molecular weight, Gd-DTPA is rapidly cleared from the blood stream. This severely limits the imaging window, the number of optimal images that can be taken after each injection, and increases the agent's required dose and relative toxicity. Moreover, such simple metal chelate image enhancers, without further modification, do not generally provide any significant site specificity.

The attachment of metal chelates to tissue or organ targeting molecules, e.g. biomolecules such as proteins, in order to produce site specific therapeutic or diagnostic agents has been widely suggested. Many such bifunctional chelating agents, i.e. agents which by virtue of the chelant moiety are capable of strongly binding a therapeutically or diagnostically useful metal ion and by virtue of the site-specific molecular component are capable of selective delivery of the chelated metal ion to the body site of interest, are known or have been proposed. However, drawbacks of conjugating metal chelates to protein carriers for use in MR imaging include inappropriate biodistribution, toxicity and short blood half-life. Their use in MR imaging is therefore limited. In addition, proteins provide a defined structure not subject to wide synthetic variation.

Site-specific uses of various imaging techniques are enhanced by the use of a multiplicity of the appropriate metal ion conjugated to a site-directed macromolecule and numerous attempts have been made to produce bifunctional polychelants with increased numbers of chelant moieties per site-specific macromolecule.

For site-specific image enhancement however it is important that the site specificity of the tissue or organ targeting moiety of such chelates of bifunctional chelants should not be destroyed by the conjugation to the targeting moiety of the chelant moiety. Where the bifunctional chelant contains only one chelant moiety this is not generally a severe problem. However, when attempts have been made to produce bifunctional polychelants by conjugating several chelant moieties onto a single site-specific macromolecule, it has been found not only may the maximum achievable chelant:site-specific macromolecule ratio be relatively limited but as the ratio achieved increases, so the site-specificity of the resulting bifunctional polychelant decreases.

In order to overcome the problems of attaching larger numbers of chelant moieties to a site-specific macromolecule without destroying its site-specificity, i.e. without disturbing its binding site(s), there have been many proposals for the use of a backbone molecule to which large numbers of chelant moieties can be attached to produce a polychelant, one or more of which can then be conjugated to the site-specific macromolecule to produce the bifunctional polychelant.

Bifunctional polychelants in which the chelant moieties are residues of open chain PAPCAs, such as EDTA and DTPA, and in which the backbone molecule is a polyamine such as polylysine or polyethyleneimine have been produced.

WO-A-90/12050 describes techniques for producing polychelants comprising macrocyclic chelating moieties, such as polylysine-polyDOTA, and for the preparation of corresponding bifunctional polychelants. This document also suggests the use of starburst dendrimers, such as a sixth generation PAMAM starburst dendrimer as the skeleton for such polychelants. WO-A-93/06868 similarly describes polychelants comprising dendrimeric backbone molecules linked to a plurality of macrocyclic chelant moieties, e.g. DOTA residues. These in turn may be conjugated to a site-directed molecule, e.g. a protein. However, to date starburst dendrimers have found little use in imaging.

Thus, there still exists a need for other polymeric contrast agents, e.g. MR, X-ray, ultrasound, light-based and nuclear, which contain relatively large amounts of metal per molecule, are of a molecular weight which enables them to be circulated within the blood for extended periods of time and which exhibit improved biodistribution.

The present invention lies in the recognition that co-polymers of amino acids carrying or attached to one or more reporter groups, e.g. chelating moieties, fluors, or absorbers, are particularly suitable for diagnostic and therapeutic use by virtue both of their structures and of their substantial uniformity in terms of molecular weight distribution. Moreover, by virtue of their relatively high molecular weights such compounds can function as effective blood pool agents without requiring attachment to site-directed biomolecules.

Thus viewed from one aspect the invention provides a compound comprising a linear, branched or dendrimeric polymer backbone with linked thereto at least one reporter moiety, said polymer backbone comprising a plurality of amine-containing acids, e.g. amino acid residues or similar non-native amine-containing acids; with the proviso that when the polymer backbone is linear, the reporter moiety comprises an iodinated contrast agent, an ultrasound contrast agent, a light-based reporter or a metal chelator other than DOTA, DTPA or similar polyaminopolycarboxylic acids. When the polymer backbone is linear, the reporter moiety preferably comprises an iodinated contrast agent or TMT.

As used herein, the term “reporter moiety” is intended to define any atom, ion or molecule which may be linked to the polymer backbone to produce an effect which is detectable by any chemical, physical or biological examination. A reporter moiety may thus be either a therapeutic or diagnostic agent, e.g. a contrast agent or pharmacologic agent. Where two or more reporter moieties are attached to a given polymer backbone, these may be identical or different. Thus, these may comprise any combination of diagnostic and/or therapeutic agents. The number of attached reporter moieties depends on the structure of the polymer backbone, in particular the degree of any branching, but generally will be in the range of from 3 to 200, preferably up to 100, e.g. up to 50.

Dendrimeric (or cascade) polymers are preferred as the backbone moiety. These are formed from monomers which act as branching sites and with each successive branching a new “generation” is formed. The dendrimeric backbone molecule preferably comprises a multiplicity of native or non-native, preferably native amino acid residues arranged to extend radially outwards from a central core moiety. These amino acid residues may be terminally bonded directly, or optionally via a linking group, to one or more reporter groups. Alternatively, these may be terminally branched by the addition of further amino acid residues. A backbone molecule wherein a central branched core has itself been terminally branched once is termed a first-generation backbone molecule. Further terminal branching of the amino acid residues of first-generation backbone molecules provides second, third, fourth etc. generation backbones. With each successive round of branching, the number of attachment points available for bonding to reporter groups increases. Depending on the nature of the central core moiety, branching from this may extend radially in one or more directions, resulting in either radially asymmetrical or symmetrical dendrimers. Preferably, the dendrimer backbone molecules are radially asymmetrical.

Dendrimeric polymers comprising a plurality of native or non-native, preferably native amino acid residues form a further aspect of the invention. Conveniently, these comprise from 3 to 200 amino acid residues, e.g. from 3 to 100 amino acid residues extending radially from a central core moiety.

Whilst the core moiety may itself comprise one or more amino acid residues, other core moieties are contemplated. Typically, the core moiety may be any molecule to which a multiplicity of successive amino acid residues may be attached and may itself comprise a reporter moiety. Suitable core moieties include

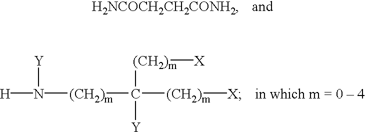

Y represents hydrogen or an alkyl or aryl group, e.g. a C₁₋₆alkyl group; and

X represents a —CO₂H, —SO₂Cl or —CH₂Br group, as well as modifications thereto and derivatives thereof.

In one embodiment of the invention, the dendrimer core may itself comprise a reporter moiety. Thus, in another aspect, the invention provides a compound comprising a dendrimeric polymer backbone extending radially from a reporter moiety, said polymer backbone comprising a plurality of amino acid residues.

Preferably, biodegradable linking groups serve to link the reporter moieties to the polymer backbone. In this way, biodegradation of the compound at the targeted site results in release of the reporter moieties, e.g. an ionic or non-ionic contrast agent at the site of interest. Examples of suitable linking groups include amide, ether, thioether, guanidyl, acetal, ketal and phosphoester groups. Linkage between the backbone and the reporter groups is preferably via an amide bond, the amide nitrogen deriving from the backbone molecule and the amide carbonyl deriving from a carboxyl or carboxyl derivative on the reporter group.

The advantage of a biodegradable polymer is that it will not accumulate at the injection site, e.g. during lymphographic procedures, or in tissues, e.g. the liver during angiographic procedures provided its degradation rate is tuned to the required imaging time. Biodegradability of the compounds of the invention can be adjusted by selection of particular linker and peptide cluster compounds. Moreover, if desired, the biodegradability of the linkers and polymer backbones can be optimised in vitro using purified enzymes and/or biological fluids/tissues. The use of amino acid monomers which themselves are rapidly cleared may further aid clearance after imaging.

Preferred polymer backbones are those comprising from 3 to 200 amino acid residues, preferably from 3 to 100 amino acid residues and having a molecular weight of from 300 to 20,000 daltons. These are preferably bonded via peptide bonds, thereby ensuring the biodegradability of the polymer and subsequent elimination from the body. The polyamino acid may be a polymer of a single species or at least two different species of amino acids, or may be a block copolymer. Preferably the polyamino acid is poly-1-aspartic acid.

Particularly preferred compounds in accordance with the invention are those of formula I:

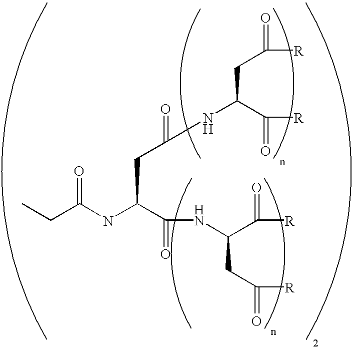

wherein n is an integer of from 1 to 100; and R represents a reporter group or a biodegradable linker-reporter adduct.

In a preferred embodiment of the invention, the reporter moieties are chelating agents. These are capable of chelating metal ions with a high level of stability, and may be metallated with the appropriate metal ion(s), e.g. to enhance images in MRI, gamma scintigraphy or X-ray or to deliver cytotoxic doses of radioactivity to kill undesirable cells such as tumors. Conveniently, the chelating agents are contrast agents comprising at least one paramagnetic metal ion. Alternatively, the chelating agents may be used in their unmetallated or undermetallated state for absorption of available metal ions in vivo, e.g. in metal detoxification.

The reporter moieties may also comprise therapeutic agents, e.g. antibiotic, analgesic, anti-inflammatory or other bioactive agents. Prolonged circulation in the blood of polymers carrying such agents substantially prolongs their therapeutic effect. Proteolysis of the linking groups provides a release of therapeutic agent. Selection of a particular linking group thus provides the potential for a timed release of therapeutic agent at the desired site of interest.

If desired, the compounds in accordance with the invention can be attached by well-known methods to one or more site-directed molecules or targeting agents, e.g. a protein, to form bifunctional polymers which can enhance images and/or deliver cytotoxic doses of radioactivity to the targeted cells, tissues, organs, and/or body ducts. Targeting of contrast agents to the site of interest in this way increases the effectiveness of the imaging method. Such agents accumulate at the site of interest which is dependent upon the specificity of the targeting agent. Alternatively, the polymers may be used as blood pool agents without being coupled to site directed molecules.

For those compounds of the invention comprising a dendrimeric backbone moiety, any terminal amino acid residues may thus be bonded either directly or via a biodegradable linking group to either a reporter or a targeting agent. Preferably, where the core moiety is itself a reporter group, each terminal amino acid residue is bound via a biodegradable linking group to a targeting agent. In this way, a compound comprising more than one targeting agent can be provided. Conveniently the number of targeting agents will be from 1 to 128, preferably from 1 to 16, e.g. from 1 to 4.

In an alternative embodiment of the invention those compounds comprising a dendrimeric polymer backbone may comprise a targeting agent or site-directed macromolecule as the core moiety. The resulting peptide cluster may in turn be linked to one or more reporter moieties. Viewed from a yet further aspect, the invention thus provides a compound comprising a dendrimeric polymer backbone extending radially from a targeting agent, said polymer backbone comprising a plurality of amino acid residues with linked thereto at least one reporter moiety.

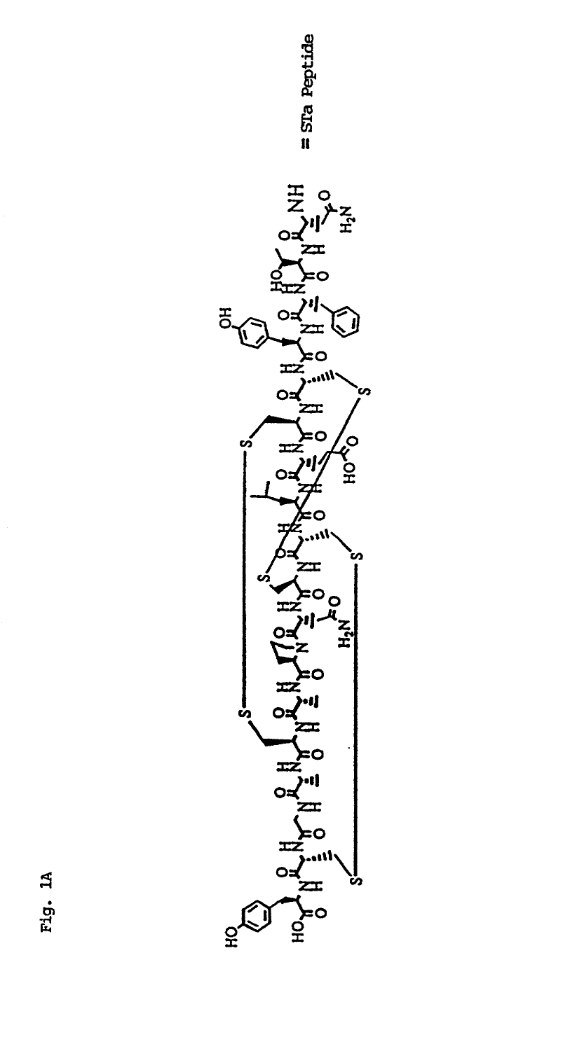

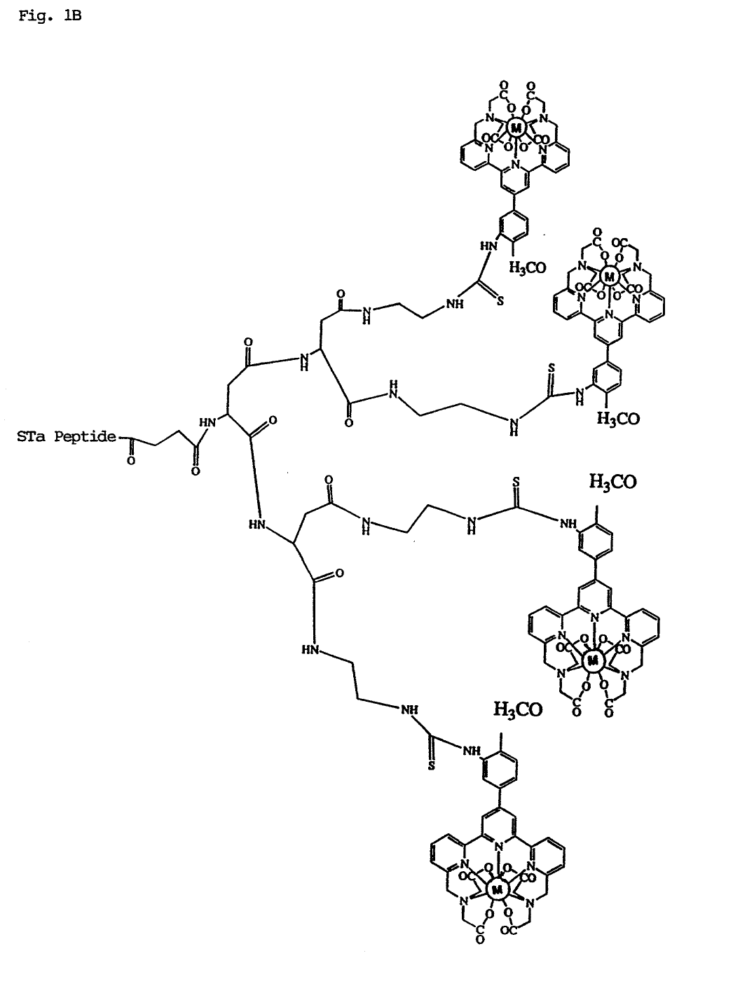

The heat stable STa enterotoxin from E. coli as described in WO-A-95/11694 is particularly suitable as a core targeting agent. Attached FIG. 1 illustrates a compound of the invention in which the STa peptide is linked to a poly-1-aspartic acid cluster (Asp3) which in turn is linked to a plurality of TMT reporter molecules.

The polymers in accordance with the invention are in and of themselves useful entities in medical diagnosis and therapy, due in part to their unique localization in the body. The size of the polymer, typically 200 to 100,000 daltons, particularly 200 to 50,000 daltons, especially 10,000 to 40,000 daltons, radically alters its biodistribution. Selection of particular linking groups and/or variations in the polyamino acid sequence also affects the biodistribution of the polymers and the attached reporter or targeting agents.

The compounds of the invention generally have extended intravascular residence times, e.g. of the order of hours, although this can be specifically tailored according to the desired use of the compounds by selection of appropriate linking agents and/or modification of the polyamino acid sequence of the backbone polymer. Usually the compounds will eventually clear into the extracellular fluid (ECF) space and undergo renal excretion. Since the compounds remain primarily in the intravascular system for a diagnostically useful residence time, they are suitable for a range of uses from blood pool and cardiac perfusion imaging, CNS tumour detection and volume determination to thrombus detection and angiography. As blood pool agents they are particularly suited to use in studies of blood flow or volume, especially in relation to lesion detection and myocardial perfusion studies. The conventional monomeric MRI contrast agents which rapidly disperse into the extracellular/extravascular space cannot readily be used for these purposes. Moreover in view of their enhanced relativity, the polymers according to the invention can be administered at significantly reduced dosages relative to current monomeric MRI contrast agents such as GdDTPA and GdDOTA, thus providing a significantly improved safety margin in their use.

The invention thus provides compounds which are able to provide MR contrast enhancement of the blood pool for long periods of time, which have a specificity towards accumulation in various body tissues, which provide relatively large amounts of metal and whose molecular weight can be synthetically tailored to produce an agent of desired composition, molecular weight and size.

Furthermore, by suitable selection of chelated species, chelates according to the invention may be produced which are capable of functioning as X-ray agents, e.g. by choosing tungsten, and also as MR contrast agents by choosing an appropriate metal ion e.g. a lanthanide ion.

Attachment of the compounds to a site-directed molecule results in even greater in vivo target specificity. The site-directed molecule is preferably an antibody, antibody fragment, other protein or other macromolecule which will travel in vivo to that site to deliver the chelated metals. In the present invention the capacity of this site-directed macromolecule to travel and/or bind to its target is not-compromised by the addition of the chelated metals. The number of chelates per molecule is sufficient to enhance the image of that particular target.

Suitable chelating agents for attachment to the polymer backbone include both linear and macrocyclic PAPCAs. Examples of suitable PAPCAs include ethylenediamine tetraacetic acid (EDTA), diethylenetriamine pentaacetic acid (DTPA), 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecanetriacetic acid (DOXA), 1,4,7-triazacyclononanetriacetic acid (NOTA) and 1,4,8,11-tetraazacyclotetradecanetetraacetic acid (TETA).

Other chelating agents suitable for attachment to the polymer backbone include terpyridines such as described in U.S. Pat. No. 5,367,080, e.g. 4′-(3-amino-4-methoxy-phenyl)-6,6″-bis(N′,N′-dicarboxymethyl-N-methylhydrazino)-2,2′:6′, 2″-terpyridine (THT) and 4′-(3-amino-4-methoxy-phenyl)-6,6″-bis[N,N-di(carboxymethyl)aminomethyl]-2,2′:6′,2″-terpyridine (TMT).

Metals that can be incorporated, through chelation, include lanthanides and other metal ions, including isotopes and radioisotopes thereof, such as, for example, Mg, Ca, Sc, Ti, B, V, Cr, Mn, Fe, Co, Ni, Cu, Zn, Ga, Sr, Y, Zr, Tc, Ru, In, Hf, W, Re, Os, Pb and Bi. The choice of metal ion for chelation will depend upon the desired therapeutic or diagnostic application.

For use in X-ray contrast imaging, the reporter moiety may comprise an ionic or non-ionic iodinated monocyclic or bis-cyclic X-ray contrast agent. By mono and bis-cyclic is meant that the contrast agents contain either one or two iodinated rings. Generally, the iodinated rings will be di- or tri-iodinated, e.g. tri-iodinated aryl rings, in particular phenyl rings. Examples of iodinated contrast agents for use in accordance with the invention include iohexol, iopentol, iopamidol and iodixanol. Conveniently, one or more iodinated contrast agents may be conjugated to form an alternating co-polymer which in turn can be attached to the polymer backbone. An example of the synthesis of such a co-polymer from iodixanol is shown below:

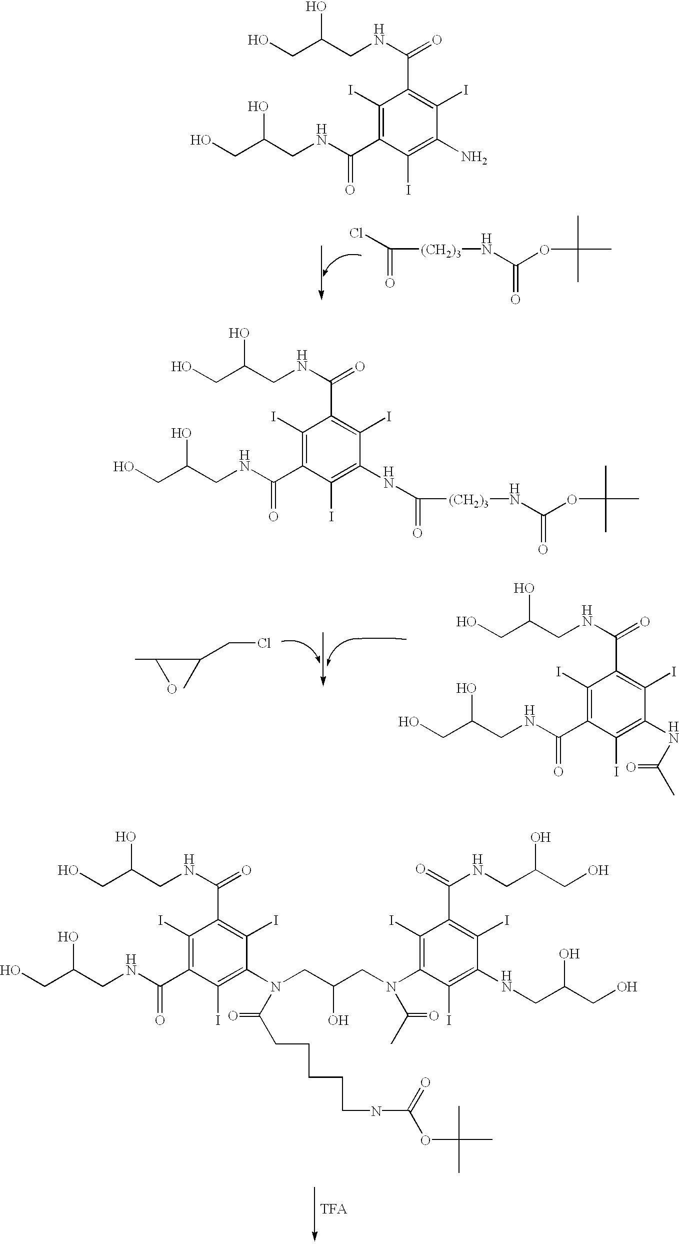

The bifunctional agents in accordance with the invention involve coupling the compounds to a site-directed molecule. The site-directed molecules may be any of the molecules that naturally concentrate in a selected target organ, tissue, cell or group of cells, or other location in a mammalian body, in vivo. These can include amino acids, oligopeptides (e.g. hexapeptides), molecular recognition units (MRU's), single chain antibodies (SCA's), proteins, non-peptide organic molecules, Fab fragments, and antibodies. Examples of site-directed molecules include polysaccharides (e.g. CCK and hexapeptides), proteins (such as lectins, asialofetuin, polyclonal IgG, blood clotting proteins (e.g. hirudin), lipoproteins and glycoproteins), hormones, growth factors, and clotting factors (such as PF4). Exemplary site-directed proteins include E.coli heat stable enterotoxin STa and its analogues, polymerized fibrin fragments (e.g., E₁), serum amyloid precursor (SAP) proteins, low density lipoprotein (LDL) precursors, serum albumin, surface proteins of intact red blood cells, receptor binding molecules such as estrogens, liver-specific proteins/polymers such as galactosyl-neoglycoalbumin (NGA) (see Vera et al. in Radiology 151: 191 (1984)) N-(2-hydroxy-propyl)methacrylamide (HMPA) copolymers with varying numbers of bound galactosamines (see Duncan et al., Biochim. Biophys. Acta 880:62 (1986)), and allyl and 6-aminohexyl glycosides (see Wong et al., Carbo. Res. 170:27 (1987)), and fibrinogen.

The site-directed protein can also be an antibody. The choice of antibody, particularly the antigen specificity of the antibody, will depend on the desired use of the conjugate. Monoclonal antibodies are preferred over polyclonal antibodies.

Human serum albumin (HSA) is a preferred protein for the study of the vascular system. HSA is available commercially from a number of sources including Sigma Chemical Co. Preparation of antibodies that react with a desired antigen is well known. Antibody preparations are available commercially from a variety of sources. Fibrin fragment E₁ can be prepared as described by Olexa et al. in J. Biol. Chem. 254:4925 (1979). Preparation of LDL precursors and SAP proteins is described by de Beer et al. in J. Immunol. Methods 50:17 (1982). The above described articles are incorporated herein by reference in their entirety.

The compounds in accordance with the invention are conveniently prepared by conjugation of a linear, branched or dendrimeric backbone comprising a plurality of amino acid residues to one or more reporter groups in a non-reactive solvent. Linkage of the reporter groups to the backbone molecule may be effected through any reactive group and standard coupling techniques are known in the art. Preferred reaction conditions, e.g. temperature, solvents etc. depend primarily on the particular reactants and can be readily determined by those skilled in the art.

Methods for metallating any chelating agents present are within the level of skill in the art. Metals can be incorporated into a chelant moiety by any one of three general methods: direct incorporation, template synthesis and/or transmetallation. Direct incorporation is preferred.

Methods for attaching the polymer backbones to antibodies and other proteins are within the level of skill in the art. Such methods are described in Pierce 1989 Handbook and General Catalog and the references cited therein, Blatter et al, Biochem., 24:1517 (1985) and Jue et al, Biochem., 17:5399 (1978).

The polymer backbone itself may be synthesised in accordance with conventional peptide synthesis techniques. Suitable methods for forming the amino acid units are described in, for example, “Synthesis of Optically Active α-Amino Acids” by Robert M. Williams (Pergamon Press, 1989). In general, the reactive side chain groups present, e.g. amino, thiol and/or carboxy, will be protected during the coupling of the individual amino acids, although it is possible to leave some side chain groups unprotected, e.g. hydroxy, primary amide groups, during the entire synthetic procedure.

The final step in the synthesis of a compound in accordance with the invention will be the deprotection of a fully protected or partly protected derivative of such a compound and such a process forms part of the invention. Thus, the present invention provides a process for producing a compound as hereinbefore described, said process comprising deprotecting a partially or fully protected derivative thereof.

In building up the peptide chain, it is in principle possible to start either at the C-terminal or the N-terminal. However, only the C-terminal starting procedure is in common use. This is due to difficulties encountered when synthesising in the N to C direction which include an unacceptably high degree of racemisation (see Konig & Geiger, Chemische Berichte 103:2024-2033, 1970).

Contrary to expectation, it has been found that the peptide compounds for use in accordance with the invention may be produced in good yield and high purity (<0.1% racemisation per step) by synthesising in the amino to carboxy direction. This method of synthesis has been found to be particularly effective in preparing the dendrimeric polymer backbones. In particular, these have been found to be more stable than those dendrimers derived from the more conventional Michael addition chemistry. Moreover, synthesising the polymer backbones in the amino to carboxy direction has been found to produce discrete polymers which are substantially non cross-linked and which have particularly low levels of racemic impurities.

Thus, in another aspect the invention further provides a process for the preparation of a compound comprising a linear, branched or dendrimeric polymer backbone with linked thereto at least one reporter moiety, said polymer backbone comprising a plurality of amino acid residues, said process comprising:

(a) stepwise linking of successive protected amino acid residues in the amino to carboxy direction to form a polymer backbone;

(b) linking the polymer backbone to one or more reporter moieties, optionally via a linking group; and

(c) deprotecting any protected group.

Thus, one can start at the N-terminal by reaction of a suitably protected derivative of, for example, aspartic acid with a suitably protected derivative of a second aspartic acid molecule. The first aspartic acid derivative will have a protected amino group and a free carboxyl group while the other reactant will have either a free or activated α-amino group and a protected carboxyl group. After coupling, the intermediate may be purified, e.g. by chromatography, and then selectively deprotected to permit addition of further amino acid residues. This procedure is continued until the required amino acid sequence is completed.

A wide range of protecting groups for amino acids are known. Suitable amine protecting groups include carbobenzoxy (Z- or Cbz), t-butoxycarbonyl (Boc-) and 9-fluorenylmethoxycarbonyl (Fmoc-). Carboxyl protecting groups which may be used include benzyl (-Bzl) and t-butyl (-tBu).

A wide range of procedures exist for removing amine- and carboxyl-protecting groups. Amine protecting groups such as Boc and carboxyl protecting groups such as -tBu may be removed simultaneously by acid treatment, e.g. with trifluoroacetic acid.

The coupling of free amino and carboxyl groups may, for example, be effected using N,N′-dicyclohexyl carbodiimide (DCC). Other coupling agents which may be used include 1-ethyl-3-(3-dimethylaminopropyl) carbodiimide (EDC) and 2-(11-H-benzotriazolyl-1-yl)-1,1,3-tetramethyluranium tetrafluoroborate (TBTU).

The coupling reactions may be effected at ambient temperatures, conveniently in a suitable solvent system, e.g. tetrahydrofuran, dimethylformamide, dimethylsulphoxide or a mixture of these solvents.

It may be convenient to carry out the peptide synthesis on a solid phase resin support. Amino acids are added stepwise to a growing peptide chain linked to an insoluble matrix, such as polystyrene beads. One advantage of this solid-phase method is that the desired product at each stage is bound to beads which can be rapidly filtered and washed so the need to purify intermediates is obviated. A number of suitable solid phase supports are known in the art, e.g. 4-hydroxy benzyl alcohol resin which has been modified to form an ester with succinic anhydride.

The compounds of the invention, especially the bifunctional polymers, may be administered to patients for imaging in amounts sufficient to yield the desired contrast with the particular imaging technique. Generally dosages of from 0.001 to 5.0 mmoles of chelated imaging metal ion per kilogram of patient bodyweight are effective to achieve adequate contrast enhancements. For most MRI applications preferred dosages of imaging metal ion will be in the range of from 0.02 to 1.2 mmoles/kg bodyweight while for X-ray applications dosages of from 0.5 to 1.5 mmoles/kg are generally effective to achieve X-ray attenuation. Preferred dosages for most X-ray applications are from 0.8 to 1.2 mmoles of the lanthanide or heavy metal/kg bodyweight.

The dosage of the compounds of the invention for therapeutic use will depend upon the condition being treated, but in general will be of the order of from 1 pmol/kg to 1 mmol/kg bodyweight.

The compounds of the present invention may be formulated with conventional pharmaceutical or veterinary aids, for example emulsifiers, fatty acid esters, gelling agents, stabilizers, antioxidants, osmolality adjusting agents, buffers, pH adjusting agents, etc., and maybe in a form suitable for parenteral or enteral administration, for example injection or infusion or administration directly into a body cavity having an external escape duct, for example the gastrointestinal tract, the bladder or the uterus. Thus the compounds of the present invention may be in conventional pharmaceutical administration forms such as tablets, capsules, powders, solutions, suspensions, dispersions, syrups, suppositories etc. However, solutions, suspensions and dispersions in physiologically acceptable carrier media, for example water for injections, will generally be preferred.

The compounds according to the invention may therefore be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized.

For MRI and for X-ray imaging of some portions of the body the most preferred mode for administering metal chelates as contrast agents is parenteral, e.g., intravenous administration. Parenterally administrable forms, e.g. intravenous solutions, should be sterile and free from physiologically unacceptable agents, and should have low osmolality to minimize irritation or other adverse effects upon administration, and thus the contrast medium should preferably be isotonic or slightly hypertonic. Suitable vehicles include aqueous vehicles customarily used for administering parenteral solutions such as Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The solutions can contain preservatives, antimicrobial agents, buffers and antioxidants conventionally used for parenteral solutions, excipients and other additives which are compatible with the chelates and which will not interfere with the manufacture, storage or use of products.

Viewed from a further aspect the invention provides a pharmaceutical composition, e.g. an image enhancing or therapeutic composition, comprising a compound of the invention together with at least one pharmaceutical carrier or excipient.

Viewed from a still further aspect the invention provides the use of a compound according to the invention or a chelate thereof for the manufacture of an image enhancing contrast medium or a therapeutic composition.

Viewed from another aspect the invention provides a method of generating an image of a human or non-human animal, especially mammalian, body which method comprises administering to said body an image enhancing amount of a compound according to the invention and thereafter generating an image e.g. an MR, X-ray, ultrasound or scintigraphic image, of at least a part of said body.

The present invention will now be further illustrated by way of the following non-limiting examples. Unless otherwise indicated, all percentages given are by weight.

### EXAMPLE 1

### Asymmetric Peptide Cluster Z-[Asp(α,γ-Asp₂(α,γ-Asp₄(α,γ-Asp₈(α,γ-Lys₁₆(α-Reporter₁₆)]

(a) Bis-α,γ-(α,γ-(tButyl)-Aspartyl)-N-Cbz-Aspartamide “Asp3 Cluster” (Compound I)

Into a 500 mL round bottom flask was added 8.5 mmoles N-Cbz-L-Aspartic acid, 10.2 mmoles N-hydroxybenzotriazole, 25 ML THF:DMF (2:1, v/v), and 10.2 mmoles EDC (1-ethyl-3-(3-dimethylaminopropyl) carbodiimide). After stirring at room temperature for 45 minutes, 20.4 mmoles of α,γ-(tButyl)-L-Aspartic acid and 25 mmoles of N,N′-diisopropylethylamine were added with stirring. After 4 hours, an additional 10.2 mmoles EDC was added and the reaction continued as above for 3 days. This slurry was worked up by aqueous extraction.

Purity: single spot on TLC, identity confirmed by MS and NMR. Yield: 44.5%.

(b) N-Cbz-Aspartamide-((α,γ-Aspartyl-(α,γ-(tButyl)-Aspartyl)) “Asp7 Cluster” (Compound II)

Into a 500 mL round bottom flask was added 10 mmoles Compound I, 95 mL chloroform:THF:acetonitrile (2.5:7:7), 36.4 mmoles N-hydroxybenzotriazole, and 36.5 mmoles DCC (N,N′-dicyclohexylcarbodiimide). After stirring at room temperature for 20 minutes, 40 mmoles of α,γ-(tButyl)-L-Aspartic acid was added and N,N′-diisopropylethylamine was added until the pH was approximately 7. After stirring at room temperature for 16 hours, the reaction was worked up by aqueous extraction.

Purity: single spot on TLC, identity confirmed by MS and NMR. Yield: 12.1%.

(c) N-Cbz-Aspartamide-((α,γ-Aspartyl-(α,γ-(tButyl)-Aspartyl)))) “Asp15 Cluster” (Compound III)

Step 1:

0.85 mmoles of Compound II was stirred in 200 mL of 95% trifluoroacetic acid (aq.) at room temperature for 8 hours. The reaction was evaporated to dryness at 40° C. in vacuo and then re-evaporated to dryness from 200 mL of toluene and then from THF.

Step 2:

Into a 250 mL round bottom flask was added 0.85 mmoles from Step 1 above, 90 mL DMF:THF (1:1, v/v), 8.12 mmoles N-hydroxybenzotriazole, and 8.12 mmoles EDC (1-ethyl-3-(3-dimethylaminopropyl)carbodiimide). After stirring at room temperature for 20 minutes, 16.24 mmoles of α,γ-(tButyl)-L-Aspartic acid and 19.92 mmoles N,N′-diisopropylethylamine were added. After stirring at room temperature for 16 hours, the reaction was worked up by aqueous extraction and ion exchange chromatography.

Purity: single spot on TLC, identity confirmed by MS and NMR. Yield: 82.2%.

(d) N-Cbz-Aspartamide-((α,γ-Aspartyl-(α,γ-Aspartyl-(Aspartyl(α,γ-Lysyl((α-methoxyethylamide, ε-amine))))))) “Asp15Lys16 Cluster” (Compound IV)

Step 1:

0.7 mmoles of Compound III was stirred in 200 mL of 95% trifluoroacetic acid (aq.) at room temperature for 8 hours. The reaction was evaporated to dryness at 40° C. in vacuo and then re-evaporated to dryness from 200 mL of toluene and then from THF.

Step 2:

Into a 250 mL round bottom flask was added 0.7 mmoles of compound from Step 1 above, 90 mL DMF:THF (1:1, v/v), 8.12 mmoles N-hydroxybenzotriazole, and 8.12 mmoles EDC (1-ethyl-3-(3-dimethylaminopropyl) carbodiimide). After stirring at room temperature for 20 minutes, 16.24 mmoles of α,γ-(tButyl)-L-Aspartic acid and 19.92 mmoles N,N′-diisopropylethylamine were added. After stirring at room temperature for 16 hours, the reaction was worked up by aqueous extraction and ion exchange chromatography.

Purity: single spot on TLC, identity confirmed by MS and NMR. Yield: 99%.

Step 3:

Into a 250 mL round bottom flask was added 0.7 mmoles of compound from Step 2 above, 100 mL DMSO:DMF:THF (1.5:3.5:5, v/v), 27.5 mmoles N-hydroxybenzotriazole, and 27.5 mmoles EDC (1-ethyl-3-(3-dimethylaminopropyl)carbodiimide). After stirring at room temperature for 20 minutes, 55 mmoles of α-BOC-L-Lysine and 68.7 mmoles N,N′-diisopropylethylamine were added. After stirring at room temperature for 16 hours, the reaction was worked up by aqueous extraction and Gel permeation chromatography.

Purity: single spot on TLC.

Step 4:

Into a 250 mL round bottom flask was added compound from Step 3 above, 40 mL DMF:DCM (2:2, v/v), 23.5 mmoles N-hydroxybenzotriazole, and 23.5 mmoles EDC (1-ethyl-3-(3-dimethylaminopropyl)carbodiimide). After stirring at room temperature for 30 minutes, 75 mmoles of 2-methoxyethanolamine were added. After stirring at room temperature overnight, the reaction was worked up by aqueous extraction and Ion Exchange chromatography.

Purity: single spot on TLC. Yield: 90%.

(e) N-Cbz-Aspartamide-((α,γ-Aspartyl-(α,γ-Aspartyl-(Aspartyl(α,γ-Lysyl((α-methoxyethylamide, ε-TMT))))))) “Asp15Lys16TMT16 Cluster” (Compound V)

Into a 250 mL round bottom flask was added Compound IV, 1.1 molar equivalents of TMT-NCS and 100 mL of 50 mM sodium borate at pH 9.0. After stirring at room temperature for 48 hours, the reaction was worked up by diafiltration (2000 MW cutoff).

Purity: 80% by RP-HPLC.

### EXAMPLE 2

### Symmetric Aspartic Acid Cluster

(a) Bis-(α,γ-(tButyl)-Aspartyl)succinamide (Compound I)

Synthetic Route A:

Into a 2 Liter round bottom flask was added 20 mmoles succinic acid, 26 mmoles EDC (1-ethyl-3-(3-dimethylaminopropyl) carbodiimide), 24 mmoles triethylamine, 12 mmoles TBTU (2-(1-H-benzotriazolyl-1-yl)-1,1,3,3-tetramethyluronium tetrafluoroborate), and 150 mL THF:DMF (2:1, v/v), then 20 mmoles α,γ-(tButyl)-L-Aspartic acid. This slurry was allowed to react for 4 days at room temperature and then worked up by aqueous extraction.

Purity: single spot on TLC, identity confirmed by MS and NMR. Yield: 23.2%.

Synthetic Route B:

Into a 2 Liter round bottom flask was added 10 mmoles succinic acid, 100 mL THF:DMF (2:1 v/v), 60 mmoles triethylamine and 20 mmoles TBTU (2-(1-H-benzotriazoyl-1-yl) -1,1,3,3-tetramethyluronium tetrafluoroborate). After 15 minutes of stirring, 22 mmoles α,γ-(tButyl)-L-Aspartic acid were added. This slurry was allowed to react for 21 hours at room temperature and then worked up by aqueous extraction.

Purity: single spot on TLC, identity confirmed by MS and NMR. Yield: 64.7%.

(b) (Bis-α,γ-Aspartyl-(α,γ-(tButyl)-Aspartyl) )-succinamide (Compound II)

Step 1:

4.6 mmoles of Compound I was stirred into 100 mL of trifluoroacetic acid/dichloromethane (1:1, v/v) at room temperature for 45 minutes. The reaction was evaporated to dryness at 30° C. in vacuo and then re-evaporated to dryness from each of five consecutive 100 mL-volumes of chloroform.

Step 2:

The product from Step 1 was dissolved in 250 mL THF:DMF (1:1, v/v) with 60 mmoles of triethylamine and 40 mmoles of L-aspartic acid-(α,γ-(tButyl)ester. To this solution was added 60 mmoles of TBTU. After 16 hours, an additional 20 mmoles of L-aspartic acid-(α,γ-(tButyl)ester was added and the reaction continued overnight.

Aqueous workup and ion-exchange chromatography yielded a single major spot on TLC which was identified as the desired compound by MS and NMR. Yield: 90%.

### EXAMPLE 3

### X-Ray Contrast Agent

(a) Synthesis of Iodinated Monomer (Compound I):

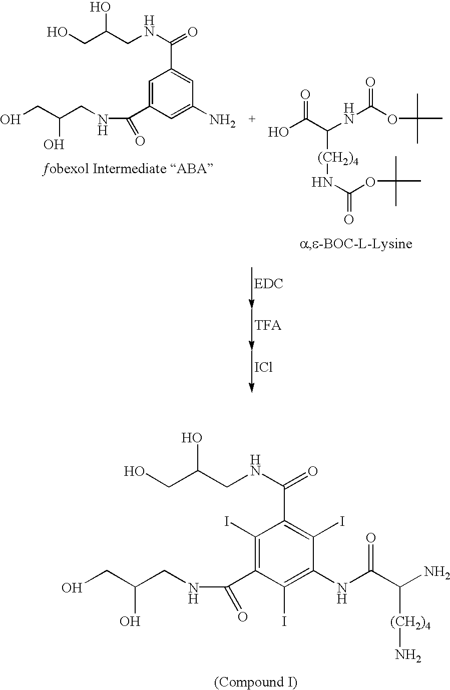

(b) Compound I may be coupled to any one of the Aspₓ clusters described in Examples 1 and 2 to form an iodinated X-ray contrast agent.

1. A compound comprising a radically asymmetric dendrimeric polymer backbone with linked thereto at least one reporter moiety, said polymer backbone comprising a plurality of amine-containing acids.

2. A compound as claimed in claim 1, wherein said polymer backbone comprises a plurality of native or non-native amino acid residues.

3. A compound as claimed in claim 2, wherein said polymer backbone comprises from 3 to 200 amino acid residues.

4. A compound as claimed in any one of claims 1 to 3, wherein said dendrimeric polymer backbone comprises from 3 to 200 amino acid residues extending radially from a central core moiety.

5. A compound as claimed in claim 4, wherein said core moiety is selected from H₂NCOCH₂CH₂CONH₂, and 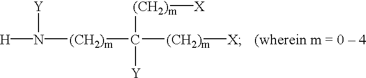
each Y independently represents hydrogen or an alkyl or aryl group; and
each X independently represents a —CO₂H, —SO₂Cl or —CH₂Br group) and derivatives thereof.

6. A compound as claimed in claim 4, wherein said core moiety comprises a reporter moiety.

7. A compound as claimed in claim 4, wherein said core moiety comprises a targeting agent capable of travelling to or binding specifically to targeted cells, tissues, organs or other locations in a mammalian body.

8. A compound as claimed in any preceding claim, wherein said polymer backbone has a molecular weight of from 300 to 20,000 daltons.

9. A compound as claimed in any one of claims 2 to 8, wherein said polymer backbone comprises a polymer of a single species or at least two different species of amino acids, or a block copolymer.

10. A compound as claimed in claim 9, wherein said polymer backbone is poly-1-aspartic acid.

11. A compound as claimed in any preceding claim comprising from 3 to 200 reporter moieties.

12. A compound as claimed in any preceding claim, wherein each reporter moiety is linked to said polymer backbone via a biodegradable linking group.

13. A compound as claimed in claim 12, wherein said linking group is selected from amide, ether, thioether, guanidyl, acetal, ketal and phosphoester groups.

14. A compound as claimed in claim 12, wherein said linking group comprises an amide bond, the amide nitrogen deriving from the backbone molecule and the amide carbonyl deriving from a carboxyl or carboxyl derivative on the reporter group.

15. A compound as claimed in any preceding claim, wherein at least one reporter moiety comprises a diagnostic or therapeutic agent.

16. A compound as claimed in claim 15, wherein said agent comprises the residue of a chelating agent or metal chelate thereof.

17. A compound as claimed in claim 16, wherein said chelating agent is a contrast agent comprising at least one paramagnetic metal ion.

18. A compound as claimed in claim 17, wherein said metal ion is selected from the lanthanide metal ions, Mg, Ca, Sc, Ti, B, V, Cr, Mn, Fe, Co, Ni, Cu, Zn, Ga, Sr, Y, Zr, Tc, Ru, In, Hf, W, Re, Os, Pb and Bi.

19. A compound as claimed in claim 16 or claim 17, wherein said chelating agent is selected from ethylenediamine tetraacetic acid (EDTA), diethylenetriamine pentaacetic acid (DTPA), 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecanetriacetic acid (DOXA), 1,4,7-triazacyclononanetriacetic acid (NOTA) and 1,4,8,11-tetraazacyclotetradecanetetraacetic acid (TETA).

20. A compound as claimed in claim 16 or claim 17, wherein said chelating agent is selected from 4′-(3-amino-4-methoxy-phenyl)-6,6″-bis(N′,N′-dicarboxymethyl-N-methylhydrazino)-2,2′:6′,2″-terpyridine (THT) and 4′-(3-amino-4-methoxy-phenyl) -6,6″-bis [N,N-di(carboxymethyl) aminomethyl]-2,2′:6′,2″-terpyridine (TMT).

21. A compound as claimed in claim 15, wherein said agent comprises an ionic or non-ionic iodinated monocyclic or bis-cyclic X-ray contrast agent.

22. A compound as claimed in any preceding claim linked to a targeting agent capable of travelling to or binding specifically to targeted cells, tissues, organs or other locations in a mammalian body.

23. A compound as claimed in claim 7 or claim 22, wherein said targeting agent comprises E. coli heat stable enterotoxin STa or an analogue thereof.

24. A dendrimeric polymer comprising a plurality of native or non-native amino acid residues extending radially asymmetrically from a central core moiety.

25. A dendrimeric polymer as claimed in claim 24, wherein said core moiety is as defined in any one of claims 5 to 7.

26. A process for preparing a compound as claimed in any one of claims 1 to 23, said process comprising conjugating at least one reporter moiety to a radially asymmetric dendrimeric polymer backbone comprising a plurality of amino acid residues.

27. A process for preparing a compound as claimed in any one of claims 1 to 23, said process comprising the step of deprotecting a partially or fully protected derivative thereof.

28. A process for the preparation of a compound comprising a linear, branched or dendrimeric polymer backbone with linked thereto at least one reporter moiety, said polymer backbone comprising a plurality of amino acid residues, said process comprising:
(a) stepwise linking of successive protected amino acid residues in the amino to carboxy direction whereby to form a polymer backbone;
(b) linking the polymer backbone to one or more reporter moieties, optionally via a linking group; and
(c) deprotecting any protected group.

29. A pharmaceutical composition comprising a compound as claimed in any one of claims 1 to 23, together with at least one pharmaceutical carrier or excipient.

30. Use of a compound as claimed in any one of claims 1 to 23 in the manufacture of an image enhancing-contrast medium or a therapeutic composition.

31. A method of generating an image of the human or non-human animal body, said method comprising the step of administering to said body a compound as claimed in any one of claims 1 to 23 and thereafter generating an image of at least a part of said body.
